(19) United States
(12) Patent Application Publication
(10) Pub. No.: US 2023/0026646 A1
(43) Pub. Date: Jan. 26, 2023

(54) METHOD FOR SCREENING THERAPEUTIC DRUG USED FOR TREATING PSORIASIS

(71) Applicant: Chung-Yang YEN, Taichung City (TW)

(72) Inventor: Chung-Yang YEN, Taichung City (TW)

(21) Appl. No.: 17/856,260

(22) Filed: Jul. 1, 2022

(30) Foreign Application Priority Data
Jul. 1, 2021 (TW) 110124202

(51) Int. Cl. G01N 33/50; G01N 33/68

(52) U.S. Cl. CPC G01N 33/5023; G01N 33/6863; G01N 2800/52

(57) ABSTRACT

A method for screening a therapeutic drug used for treating psoriasis includes the steps of: (a) isolating and separating fresh peripheral blood mononuclear cells (PBMC); (b) activating the peripheral blood mononuclear cells with a stimulant to cause the cells to produce IFN-γ and RANTES; (c) administering the plurality of candidate drugs to the activated peripheral blood mononuclear cells; (d) detecting expression levels of IFN-γ and RANTES in the peripheral blood mononuclear cells administered with the plurality of candidate drugs; (e) recognizing and removing a disqualified drug from the plurality of candidate drugs according to a variation degree of the expression level of RANTES; and (f) screening the therapeutic drug from the plurality of the candidate drugs excluding the disqualified drug according to the expression level of IFN-γ.

### CROSS-REFERENCE TO RELATED APPLICATION

This application claims the priority benefit of Taiwan patent application serial no. 110124202, filed Jul. 1, 2021. The entirety of the above-mentioned patent application is hereby incorporated by reference herein and made a part of this specification.

### BACKGROUND

### Field of the Invention

The invention generally relates to psoriasis treatment. More specifically, the invention relates to a drug screening method for selecting a drug from multiple candidate drugs to treat psoriasis.

### Description of the Related Art

Psoriasis is a chronic inflammatory skin disease mediated by immunity. It is generally believed that psoriasis is related to immune dysfunction and genetics. However, other factors such as seasonal changes, stress, viruses, bacteria, metabolic disorders, drug-induced, trauma and diet are also considered to have the potential to induce psoriasis. The typical symptoms of psoriasis are lumpy, abnormal skin lesions that appear on the surface of the patient's skin, with redness, itching, and scaling in the affected area. The prevalence of psoriasis is known to be about 24%, women and men are equally affected, and the prevalence is higher in adults than children (about 0-2.1%).

Current treatment methods for psoriasis may include topical therapy, phototherapy, photochemotherapy and systemic therapy. For topical therapy, drugs such as steroid, Calcipotriol, Anthralin, or Coal tar are used locally on the affected area. Phototherapy uses ultraviolet rays to irradiate the skin of patients. Photochemotherapy refers to a combined therapy of oral psoralen and ultraviolet irradiation with a wavelength of 320-400 nm. Systemic therapy uses biological agents (e.g. therapeutic antibodies), cyclosporine, methotrexate and tretinoin to treat psoriasis. Although there are various methods for the treatment of psoriasis, it is often found that, due to the different immune responses of individual patients, one patient responds well to this drug but another patient does not. Therefore, it is not easy for clinicians to find a suitable therapy for individual patients from the above-mentioned therapies, which causes low patient's compliance with treatment and waste of medical resources.

### BRIEF SUMMARY OF THE INVENTION

The invention provides a drug screening method to select a therapeutic drug suitable for an individual psoriasis patient from a plurality of candidate drugs, so as to treat the psoriatic disease of the patient.

As embodied and broadly described in the present disclosure, one aspect of the present disclosure relates to a method for screening a therapeutic drug from a plurality of candidate drugs, where the therapeutic drug is used for treating a patient suffering from psoriasis. The method includes the following steps:

(a) isolating and separating fresh peripheral blood mononuclear cells (PBMC);

(b) activating the peripheral blood mononuclear cells with a stimulant to cause the cells to produce interferon-gamma (IFN-γ) and, regulated on activation, normal T cell expressed and secreted (RANTES);

(c) respectively administering the plurality of candidate drugs to the activated peripheral blood mononuclear cells;

(d) detecting expression levels of IFN-γ and RANTES in the peripheral blood mononuclear cells administered with the plurality of candidate drugs;

(e) recognizing and removing a disqualified drug from the plurality of candidate drugs according to a variation degree of the expression level of RANTES;

(f) screening the therapeutic drug from the plurality of the candidate drugs excluding the disqualified drug according to the expression level of IFN-γ.

According to another aspect of the invention, a method for screening a therapeutic drug from a plurality of candidate drugs used for treating psoriasis in an individual includes the following steps:

(a) isolating and separating fresh peripheral blood mononuclear cells (PBMC);

(b) activating the peripheral blood mononuclear cells with a stimulant to cause the cells to produce interferon-gamma (IFN-γ), interleukin-4 (interleukin-4, IL-4), interleukin-9 (interleukin-9, IL-9) and, regulated on activation, normal T cell expressed and secreted (RANTES);

(c) respectively administering the plurality of candidate drugs to the activated peripheral blood mononuclear cells;

(d) detecting expression levels of IFN-γ, IL-4, IL-9 and RANTES in the peripheral blood mononuclear cells administered with the plurality of candidate drugs;

(e) recognizing and removing a disqualified drug from the plurality of candidate drugs according to a variation degree of the expression level of RANTES, wherein the disqualified drug is recognized by that the expression level of RANTES detected in step (d) is increased by at least 100% than the expression level of RANTES after streptococcus activation in step (b); and

(f) screening the therapeutic drug from the plurality of the candidate drugs excluding the disqualified drug according to a ratio of the IFN-γ expression level to the IL-4 expression level or according to a ratio of the IFN-γ expression to the IL-9 expression level.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIGS. 1 and 2 graphically depicts the improvement of treatment with adalimumab selected according to the drug screening method according to an embodiment of the invention, wherein FIG. 1 shows a patient's skin condition before the treatment with adalimumab, and FIG. 2 shows the patient's skin condition after the treatment with adalimumab.

FIG. 3 shows clinical pictures of patients with drug-induced paradoxical psoriatic arthritis.

FIG. 4 shows an exemplary flow chart of the drug screening method according to an embodiment of the invention.

### DETAILED DESCRIPTION OF THE INVENTION

In order to make the description of the present disclosure more detailed and complete, an illustrative description is provided below with respect to the embodiments and specific embodiments of the present invention; but this is not the only form of implementing or using the specific embodiments of the present invention. The features of various specific embodiments as well as method steps and orders for constructing and operating these specific embodiments are encompassed in the detailed description. However, other embodiments may also be utilized to achieve the same or equivalent function and sequence of steps.

### I. DEFINITION

In this specification and the appending claims, the singular form “a” (or an) includes plural references unless the context dictates otherwise. In addition, in this specification and the appending claims, expressions such as “at least one” and “one or more” have the same meaning, and both mean that one, two, three or more are included. Furthermore, in this specification and the appending claims, each expression of “at least one of A, B and C”, “at least one of A, B or C” and “at least one of A, B and/or C” means any one of the following situations: only A, only B, only C, both A and B, both B and C, both A and C, and A, B and C.

Notwithstanding the numerical ranges and parameters setting broader scopes of the invention are only approximate, the numerical values set forth in the examples have been presented as precisely as possible. Any numerical value, however, inherently contains the standard deviation resulting from individual testing methods. As used herein, the term “about” generally refers to an actual value within plus or minus 10%, 5%, 1%, or 0.5% of a particular value or range. Alternatively, the word “about” means that the actual value lies within an acceptable standard error of the average value, as considered by one of ordinary skill in the art to which this invention pertains. Except for experimental examples, or unless otherwise expressly stated, all ranges, quantities, values and percentages used herein (e.g. material amounts, time durations, temperatures, operating conditions, quantity ratios and others) can be understood as modified by “about”. Therefore, unless otherwise stated to the contrary, the numerical parameters disclosed in this specification and the appending claims are approximate numerical values and may be changed as required. At a minimum, these numerical parameters should be construed to mean the number of significant digits indicated and the numerical values obtained by applying ordinary rounding. Numerical ranges are expressed herein as being from one endpoint to the other endpoint or between two endpoints; unless otherwise indicated, the numerical ranges described herein are inclusive of the endpoints.

As used herein, the term “treatment (or treating)” may refer to a curative or palliative measure. Specifically, as used herein, the term “treatment” refers to the administration or administration of an effective amount of a therapeutic agent described in the present disclosure, the therapeutic agent being screened using the drug screening methods described in the present disclosure, and the individual suffers from psoriasis, suffers from symptoms associated with psoriasis, a disease or disorder of psoriasis whereby partial or complete alleviate, ameliorate, relieve, delay onset, inhibit progression, reduce severity, and/or reduce the incidence of one or more symptoms or features of psoriasis.

As used herein, the term “activate (activating, or activation) refers to the use of a specific substance (e.g. streptococcus) to stimulate a living or living cell (e.g. peripheral blood mononuclear cells), causing the living body or living cells induced by the specific substance to produce a corresponding immune response (for example, secrete related inflammatory cytokines such as interferon-γ, interleukin-4, interleukin-9, etc.)

As used herein, the terms “administered,” “administering,” or “administration” are used interchangeably herein and refer to the direct contact of a target (e.g. peripheral blood mononuclear cells) with a specific substance (e.g. a candidate drug described in the present disclosure); or, the act of administering a specific substance (e.g. a therapeutic drug described in the present disclosure) to an individual (e.g. a human). For example, a drug candidate described in the present disclosure is directly added to a culture dish containing peripheral blood mononuclear cells for co-cultivation for a period of time. Furthermore, when a therapeutic agent described in the present disclosure is administered to an individual, it is meant via oral, intracranial, intraspinal, intrathecal, intramedullary, intracerebral, intracerebroventricular, intravenous, intraarterial, intracardiac, intradermal, subcutaneous, transdermal, intraperitoneal, or intramuscular routes to administer the therapeutic agents described in the present disclosure to the individual in need.

As used herein, the term “an effective amount” refers to an amount effective, in the dosage and for the time period necessary, to achieve the desired therapeutic effect (e.g., treatment of psoriasis). For therapeutic purposes, an effective amount also means that the therapeutic benefits of a component of a drug outweigh the toxic or detrimental effects of that component. An effective amount of an agent does not necessarily cure the disease or disorder, but delays, retards or prevents the development of the disease or disorder, or alleviates the symptoms associated with the disease or disorder. An effective amount can be divided into one, two or more doses and administered one, two or more times over a specified period in the appropriate dosage form. The specific effective amount depends on a variety of factors, for example, the condition to be treated, the physiological condition of the individual (e.g. the individual's weight, age, or sex), the species being treated, the duration of treatment, and concurrent therapy, the specific dosage form used, and the structure of the compound or its derivatives. An effective amount may be expressed in any suitable manner. For example, an effective amount of an agent can be expressed as the total weight of the drug (e.g. grams, milligrams, or micrograms), or as a ratio of the weight of the drug to body weight (e.g. milligrams per kilogram of body weight (mg/kg, mg/kg) Kg)). Alternatively, an effective amount of an agent can be expressed as a concentration, e.g. molar concentration, mass concentration, volume concentration, molality, molar fraction (mole fraction, mass fraction and mixing ratio. Appropriate doses range from 0.01 mg to 100.0 mg per kilogram of body weight. As will be appreciated, a wide range of adjustments in the desired dosage are also contemplated, depending upon the different compositions and the different efficacies resulting from the different routes of administration. For example, oral administration is expected to require higher doses than intravenous injection. Those skilled in the art fully understand that the dosage can be adjusted according to rules of thumb. One of skill in the art can convert doses obtained based on experimental animal models into human equivalent doses (HEDs) of drugs (e.g. therapeutic drugs described in the present disclosure). For example, those skilled in the art can use the “Estimating the Maximum Safe Starting Dose of Adult Healthy Volunteers in Initial Clinical Treatment Tests” published by the US Food and Drug Administration (FDA) (U.S. Food and Drug Administration, FDA). Safe Starting Dose in Initial Clinical Trials for Therapeutics in Adult Healthy Volunteers) to estimate the highest safe dose for human use.

As used herein, the terms “subject” or “patient” are used interchangeably to refer to an animal, including humans, that can be treated with a therapeutic agent described in the present disclosure, wherein the therapeutic agent Refers to those screened by the screening method described in the present disclosure. Unless one of the genders is specified, the terms “individual” and “patient” refer to both males and females. Accordingly, the terms “individual” and “patient” include any mammal that would benefit from treatment with the therapeutic agents described in the present disclosure. Examples of “individuals” and “patients” that can be treated with therapeutic agents described in the present disclosure include, but are not limited to, humans, rats, mice, guinea pigs, monkeys, pigs, goats, cows, horses, dogs, cats, Birds and Chickens. In an exemplary embodiment, the individual is a mouse. In another illustrative embodiment, the individual is a human.

The term “pharmaceutically acceptable” refers to molecular entities and compositions that are “generally regarded as safe,” e.g. that are physiologically tolerable when administered to human, it generally does not produce allergic reactions or similar adverse reactions, such as nausea, dizziness, and the like. Preferably, the term “pharmaceutically acceptable” as used herein means approved by a regulatory agency of the federal or state government or listed in the US Pharmacopeia or other generally recognized pharmacopeia for use in animals, particularly is for humans.

### II. DETAILED DESCRIPTION OF THE INVENTION

The purpose described in the present disclosure is to provide a method for screening out a drug suitable for the treatment of psoriasis from a plurality of candidate drugs, and the method can screen out a therapeutic drug suitable for the patient according to respective constitutions of individual psoriasis patients. Therefore, the efficacy and safety of psoriasis treatment through therapeutic drugs are improved and waste of medical resources is reduced.

Methods for Screening Drugs

Accordingly, one aspect described in the present disclosure relates to a method for screening a therapeutic drug, wherein the therapeutic drug is selected from a plurality of candidate drugs and used to treat psoriasis in a patient. The screening method according to an embodiment of the invention includes the following steps:
(a) isolating and separating fresh peripheral blood mononuclear cells (PBMC);
(b) activating the peripheral blood mononuclear cells with a stimulant to cause the cells to produce interferon-gamma (IFN-γ), interleukin-4 (interleukin-4, IL-4), interleukin-9 (interleukin-9, IL-9), interleukin-13 (interleukin-13, IL-13) and, regulated on activation, normal T cell expressed and secreted (RANTES);
(c) respectively administering the plurality of candidate drugs to the activated peripheral blood mononuclear cells;
(d) detecting expression levels of IFN-γ, IL-4, IL-9, IL-13 and RANTES in the peripheral blood mononuclear cells administered with the plurality of candidate drugs;
(e) recognizing and removing a disqualified drug from the plurality of candidate drugs according to a variation degree of the expression level of RANTES, wherein the disqualified drug is recognized by that the expression level of RANTES detected in step (d) is increased by at least 100% than the expression level of RANTES after streptococcus activation in step (b); and
(f) screening the therapeutic drug from the plurality of the candidate drugs excluding the disqualified drug according to a ratio of the IFN-γ expression level to the IL-4 expression level, a ratio of the IFN-γ expression to the IL-9 expression level, or a ratio of the IFN-γ expression level to the IL-13 expression level.

The screening method according to another embodiment of the invention includes the following steps:

(a) isolating and separating fresh peripheral blood mononuclear cells (PBMC);

(b) activating the peripheral blood mononuclear cells with a stimulant to cause the cells to produce interferon-gamma (IFN-γ) and, regulated on activation, normal T cell expressed and secreted (RANTES);

(c) respectively administering the plurality of candidate drugs to the activated peripheral blood mononuclear cells;

(d) detecting expression levels of IFN-γ and RANTES in the peripheral blood mononuclear cells administered with the plurality of candidate drugs;

(e) recognizing and removing a disqualified drug from the plurality of candidate drugs according to a variation degree of the expression level of RANTES; and

(f) screening the therapeutic drug from the plurality of the candidate drugs excluding the disqualified drug according to the expression level of IFN-γ.

The method according to another embodiment of the invention includes the following steps:

(a) isolating and separating fresh peripheral blood mononuclear cells (PBMC);

(b) activating the peripheral blood mononuclear cells with a stimulant to cause the cells to produce interferon-gamma (IFN-γ), interleukin-4 (interleukin-4, IL-4), interleukin-9 (interleukin-9, IL-9) and, regulated on activation, normal T cell expressed and secreted (RANTES);

(c) respectively administering the plurality of candidate drugs to the activated peripheral blood mononuclear cells;

(d) detecting expression levels of IFN-γ, IL-4, IL-9 and RANTES in the peripheral blood mononuclear cells administered with the plurality of candidate drugs;

(e) recognizing and removing a disqualified drug from the plurality of candidate drugs according to a variation degree of the expression level of RANTES; and

(f) screening the therapeutic drug from the plurality of the candidate drugs excluding the disqualified drug according to a ratio of the IFN-γ expression level to the IL-4 expression level or according to a ratio of the IFN-γ expression to the IL-9 expression level.

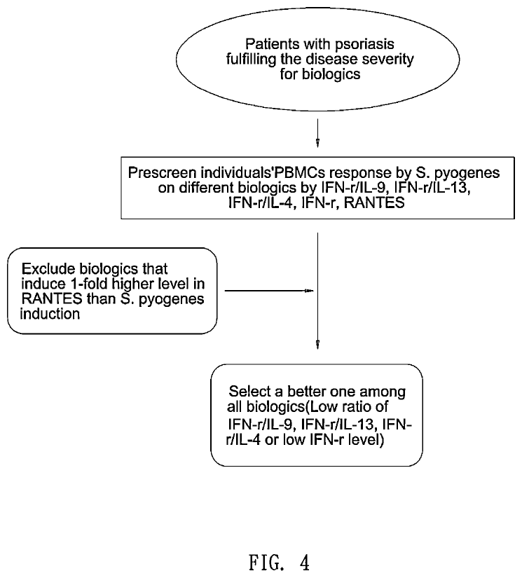

As illustrated in FIG. 4, first, in step (a), a whole blood sample is collected from a patient suffering from psoriasis, and peripheral blood mononuclear cells are isolated from the whole blood sample. Next, the obtained peripheral blood mononuclear cells are treated with a stimulus for a period of time (e.g. 16 to 72 hours, such as: 16, 18, 24, 36, 48, 60 or 72 hours; preferably, 24 hours), so that the peripheral blood mononuclear cells are activated by the stimulation of the streptococcus and secrete interferon-gamma, interleukin-4, interleukin-9, and other inflammatory cytokines (e.g. interleukin-6, interleukin-8, interleukin-13, or interleukin-17, RANTES etc.)

According to embodiments described in the present disclosure, the stimulus is a gram-positive bacteria, a gram-negative bacteria, or a lipopolysaccharide. Exemplary Gram-positive bacteria include, but are not limited to, Bacillus (e.g. Bacillus cereus, Bacillus thuringiensis, Bacillus anthracis); Listeria (Listeria) (e.g. Listeria monocytogenes); Staphylococcus (e.g. Staphylococcus aureus, Staphylococcus albus, Staphylococcus citrate (Staphylococcus citreus); Streptococcus (described below), Enterococcus (described below); and Clostridium (e.g. Clostridium botulinum, Clostridium butyricum) (Clostridium butyricum, Clostridium difficile, Clostridium perfringens, Clostridium tetani). In a preferred embodiment, the gram-positive bacteria are streptococcus.

According to one embodiment described in the present disclosure, the stimulator is a Gram-negative bacteria, including, but not limited to, Acinetobacter (e.g. Acinetobacter baumannii), calcium acetate Acinetobacter calocoaceticus, Acinetobacter lwoffi); Bdellovibrio (e.g. Bdellovibrio bacteriovorus); Enterobacter (e.g. Enterobacter carcinogens) (Enterobacter cancerogenous, Enterobacter cloacae, Enterobacter cowanii, Enterobacter gergoviae, Enterobacter taylorae); Escherichia (e.g. Escherichia coli); Haemophilus (e.g. Haemophilus ducreyi, Haemophilus influenzae); Helicobacter (e.g. Helicobacter pylori); Klebsiella (e.g. Klebsiella oxytoca, Klebsiella pneumoniae); Legionella (e.g. Legionella Legionella pneumophila); Moraxella (e.g. Moraxella catarrhalis); Neisseria (e.g. Neisseria gonorrhoeae, Neisseria meningitidis (Neisseria meningitidis); Proteus (e.g. Proteus mirabilis); Pseudomonas (e.g. Pseudomonas aeruginosa), Pseudomonas oryzihabitans, Pseudomonas plecoglossicid a)); Salmonella (e.g. Salmonella bongori, Salmonella enteritidis, Salmonella typhi); Serratia (e.g. Serratia marcescens); Shigella (e.g. Shigella boydii, Shigella dysenteriae, Shigella flexneri, Shigella flexneri (Shigella sonnei); Stenotrophomonas (e.g. Stenotrophomonas maltophilia). In a preferred embodiment, the gram-negative bacteria are Escherichia coli.

In step (b), after peripheral blood mononuclear cells and streptococcus are common cultured for a period of time (e.g. 24 hours), the culture supernatant of the cells is collected to analyze the activation of the cells to secrete inflammatory cytokines. The expression of inflammatory cytokines can be detected using a variety of assays well known to those skilled in the art, for example, an enzyme linked immunosorbent assay (ELISA) (e.g., multiplex assay), radioimmunoassay (RIA), immunofluorescence (IFA), Western blot (WB), immunoblotting (IB), immunoprecipitation (immunoprecipitation), IP), or flow cytometry (flow cytometry), or aptamer-linked immobilized sorbent assay (aptamer-linked immobilized sorbent assay) and other analytical methods. According to an operational embodiment described in the present disclosure, ELISA is used to analyze the expression of inflammatory cytokines in the activated peripheral blood mononuclear cells

Alternatively, peripheral blood mononuclear cells activated by Streptococcus to express the expression of inflammatory cytokines can be detected. The activated peripheral blood mononuclear cells can be collected, and the expression level of inflammatory cytokines can be detected by an analysis method for analyzing gene expression level. The analytical methods for analyzing gene expression are well known to those skilled in the art, including, but not limited to, genome-wide expression profiling using expressed sequence tag (EST) analysis Sequence analysis of gene expression (serial analysis of gene expression, SAGE); cDNA microarray (cDNA microarray); massively parallel signature sequencing (MPSS); RNA sequencing (RNA sequencing, RNA-seq); polymerase chain reaction (PCR) (for example, reverse transcription-PCR (RT-PCR), real-time RT-PCR or qRT-PCR), digital polymerase chain reaction (digital-PCR or dPCR), touchdown PCR (touchdown PCR), nested PCR (nested PCR), multiplex PCR (multiplex PCR), recovery condition PCR (reconditioning PCR), etc.); two-dimensional coagulation Gel electrophoresis (two-dimensional gel electrophoresis, 2-D electrophoresis); tissue array (tissue array); immunohistochemistry (immunochemistry, IHC) staining, etc.

Next, after the peripheral blood mononuclear cells are activated, the plurality of drug candidates is tested using the activated peripheral blood mononuclear cells (step (c)). The specific step is to administer the drug candidates to be tested to the activated peripheral blood mononuclear cells, respectively, so that the activated peripheral blood mononuclear cells and the drug candidates to be tested are co-cultured for a period of time (for example, 16 to 72 hours, such as: 16, 18, 24, 36, 48, 60 or 72 hours; preferably, treatment for 24 hours). For the specific concentration of the candidate drug administered to the cells, reference may be made to the trough serum concentration under steady state recommended in the relevant pharmacokinetics section in the copy list of each candidate drug. For example, adalimumab (HUMIRA®) at 4 μg/ml; golimumab (SIMPONI®) at 0.5 μg/ml; guselkumab (TREMFYA®) at 1.2 μg/ml; ixekizumab (TALTZ®) at 3.5 μg/ml; secukinumab (COSENTYX®) at 16.7 or 34 μg/ml; ustekinumab (STELARA®) at 0.25 μg/ml; brodalumab (16 μg/ml) and etanercept (1.9 μg/ml).

The plurality of candidate drugs may be current drugs for the treatment of psoriasis, or potential drugs under development for the treatment of psoriasis. According to certain embodiments described in the present disclosure, the plurality of drug candidates comprises oral A-acid, alfacept, allionol, apremilast, apremilast, acetaminophen, betamethasone, carbomer alkene, calcipotriol, calcitriol, becloxone, tar, cyclosporine, diosgenol, etanercept, fluocinolone, hydrocortisone, infliximab, sterilization tablets, pimecrolimus, tacrolimus, tazarotene, ALX-0761, BCD-085, pembrolizumab, bordalumab, CJM112, CNTO 6785, COVA322, ixekizumab, LY3114062, MSB0010841, NI-1401, perelizumab, rendolizumab, RG7624, secukinumab, vonazumab, blacumab, guselkumab, milizumab, resal Zizumab, tiltaximab, ustekinumab, adalimumab, certolizumab, etanercept, golimumab, infliximab, and/or lena chypre. In addition, in a specific embodiment, the plurality of drug candidates comprises adalimumab, golimumab, guselkumab, bordalumab, ixekizumab, etanercept, secukinumab, and ustekinumab

In step (d), after the treatment of the specific drug candidate, the culture supernatant of the cells or the cells are collected respectively, and the inflammatory cells caused by the influence of the drug on the cells are analyzed by the above analysis method to measure variations in cytokine expression levels, especially about expression variations of interferon-γ, interleukin-4, interleukin-9, interleukin-13, RANTES (regulated on activation, normal T cell expressed and secreted), etc.

In step (e), any candidate drug that may induce psoriasis arthritis (referred to as a disqualified drug) is recognized and removed from the candidate list in advance. In one embodiment, the disqualified drug can be determined by that the expression level of RANTES detected in step (d) is increased by at least 100% than the expression level of RANTES after streptococcus activation in step (b).

In step (f), variations in the expressions of interferon-γ, interleukin-4, interleukin-9 or interleukin-13 detected in step (d) are used to screen out a therapeutic drug from the plurality of candidate drugs. The variations in the expression amounts of IFN-γ, IL-4 IL-9 or IL-13 can be obtained by comparing variation rates of expressed amounts of IFN-γ, IL-4 IL-9 or IL-13 in step (d) (i.e., after administration of a specific drug candidate) to variation rates of expression amounts of IFN-γ, IL-4 IL-9 or IL-13 in step (b) (i.e., before administration of the specific drug candidate). The relevant calculation formulas (I)-(IV) for statistical analysis are listed below.

$$( IFN - γ / IL - 4 w / S . pyogenes \& biologics ) - ( IFN - γ / IL - 4 w / S . pyogenes only ) \qquad formula ( I )$$

$$\frac{\begin{matrix}( IFN - γ / IL - 4 w / S . pyogenes \& biologics ) - \\ ( IFN - γ / IL - 4 w / S . pyogenes only )\end{matrix}}{( IFN - γ / IL - 4 w / S . pyogenes only )} \qquad formula ( II )$$

$$( IFN - γ / IL - 9 w / S . pyogenes \& biologics ) - ( IFN - γ / IL - 9 w / S . pyogenes only ) \qquad formula ( III )$$

$$\frac{\begin{matrix}( IFN - γ / IL - 9 w / S . pyogenes \& biologics ) - \\ ( IFN - γ / IL - 9 w / S . pyogenes only )\end{matrix}}{( IFN - γ / IL - 9 w / S . pyogenes only )} \qquad formula ( IV )$$

The reduction rate of IFN-γ/IL-4 after treatment with biologics can be calculated by (compared with S. pyogenes induction only):

$$\frac{( IFN - γ / IL - 4 w / S . pyogenes \& biologics ) - ( IFN - γ / IL - 4 w / S . pyogenes only )}{( IFN - γ / IL - 4 w / S . pyogenes only )}$$

The difference on IFN-γ/IL-4 after treatment with biologics can be calculated by (compared with S. pyogenes induction):

(IFN-γ/IL-4 w/S. pyogenes & biologics)−(IFN-γ/IL-4 w/S. pyogenes only)

Moreover, the reduction rate and difference for IFN-γ/IL-9 IFN-γ/IL-13 or can be similarly estimated according the above formulas.

According to another operational embodiment, the peripheral blood mononuclear cells of a specific psoriasis patient (G) are treated with ixekizumab, ustekinumab, and secukinumab, wherein ixekizumab is administered The ratio of interferon-γ expression to interleukin-4 expression in cells is increased by monoclonal antibody to 6.97, while administration of ustekinumab increased the expression of IFN-γ in cells and IL-4 expression in cells The ratio of expression amount is 8.94; while administration of secukinumab resulted in a ratio of IFN-γ expression to IL-4 expression in the cells of 7.38, in this example, ixekizumab is the smallest, so ixekizumab is selected to treat the psoriasis patient.

The Psoriasis Area Severity Index (PASI) is used to evaluate the treatment effect. The evaluation method of the PASI covers the evaluation of the psoriasis area (Area) and the psoriasis severity (Severity), as shown in Table 1 and Table 2. After evaluating the psoriasis area and psoriasis severity, the value PASI can be obtained by the following formula:

PASI=0.1×(Eh+Ih+Dh)×Ah+0.3×(Et+It+Dt)×At+0.2×(Eu+Iu+Du)×Au+0.4×(El+Il+Dl)×Al; parts: head (h), trunk (t), upper limb (u), lower limb (l).

TABLE 1
Area: percentage of affected area (%)

| | percentage of affected area | | | | | | |
|---|---|---|---|---|---|---|---|
| | 0% | <10% | 10-29% | 30-49% | 50-69% | 70-89% | 90-100% |
| | Score | | | | | | |
| Location | 0 | 1 | 2 | 3 | 4 | 5 | 6 |

Location: Head(h) [symbol] trunk(t) [symbol] upper extremity(u) [symbol] lower extremity(l)

TABLE 2
Severity:

| Severity | Absent | Mild | Moderate | Severe | Most Severe |
|---|---|---|---|---|---|
| Score | 0 | 1 | 2 | 3 | 4 |
| Erythema(E) | nil | mild | pinkish | red | Dark |
| | | | | | red/purplish |
| Induration(I) | nil | Mild | Moderate | Severe | Most severe |
| | | induration | induration | | |
| Scales(D) | nil | Mild | Moderate | Severe | Most severe |
| | | scales | scales | | |

### Embodiments

Materials and Methods

1. Patients Clinical Response

Total 11 psoriasis patients agree to participate in this project and signed the written informed consent, and the study protocol is approved by the Institutional Review Board in of Taichung Veterans General Hospital (TCVGH-CE16265B; TCVGH-CE20043B) in Taiwan.

2. Cell Culture

To prepare the PBMC culture, 16 mL of blood is obtained from each patient and collected in sodium citrate tubes (Vacutainer CPT, BD, USA). Then, PBMCs are purified using centrifugation over a density gradient. The cells are washed with phosphate-buffered saline and subsequently cultured in RPMI-1640 supplemented with 10% fetal bovine serum and 1% penicillin/streptomycin at 37° C. and 5% CO₂.

A total of 6×10⁵ cells per milliliter are then cultured in a 12-well plate and treated for 24 hours with the following biologics: control, S. pyogenes only, S. pyogenes+adalimumab (4 μg/mL), S. pyogenes+golimumab (0.5 μg/mL), S. pyogenes+ustekinumab (0.25 μg/mL), S. pyogenes+ixekizumab (3.5 μg/mL), S. pyogenes+secukinumab (16.7 μg/mL), S. pyogenes+secukinumab (34 μg/mL), or S. pyogenes+guselkumab (1.2 μg/mL). The concentrations of the biological agents we tested are the trough serum concentrations at steady state indicated in the pharmacokinetic section of the reference list. The two concentrations of secukinumab (16.7 or 34 μg/mL) reflect the two common clinical doses of 150 mg or 300 mg per month, respectively. Type A streptococcus is used with McFarland 2 solution diluted 1 to 100 times the concentration (each type A streptococcus has different characteristics) for PBMC induction and different biologics drug candidates after co-incubation for 24 hours. Supernatants are collected for the subsequent measurement of cytokine levels.

3. Statistical Analysis

All statistical analyses are performed using SPSS version 22 (IBM, Armonk, N.Y., USA). Cytokine expression and PASI is analyzed with Spearman's rho. Data are presented as the mean±standard deviation. Two-sided p values of 0.05 or less are considered to indicate statistical significance.

Results of PASI Before and After Biologics Treatment and Cytokine and Chemokine Levels

Because different patients with psoriasis respond differently to different biological agents, it is critical to develop a method of evaluating medication for each patient with psoriasis to assess the appropriate therapy for that patient. The invention therefore develops the use of the expression variations in the ratio of inflammatory cytokines or inflammatory cytokines produced by peripheral blood mononuclear cells before and after administration as a medication index to assess whether the patient is suitable for a specific therapy. In this example, the relationship between changes in PASI and changes in the expression of inflammatory cytokines (i.e., ratios or differences) in different patients before and after treatment with a specific biologic is analyzed.

### Embodiment 1, IL-4

TABLE 3
PASI and IFN-γ/IL-4 ratio

| | Experienced | PASI | PASI | PASI reduction | PASI reduction |
|---|---|---|---|---|---|
| Pt | Biologies | (Before) | (After) | ratio | difference |
| A | TALTZ ® | 15 | 1.2 | −92.0% | −13.8 |
| A | STELARA ® | 16 | 0 | −100.0% | −16 |
| B | HUMIRA ® | 15 | 16 | 6.7% | 1 |
| B | STELARA ® | 15 | 0.2 | −98.7% | −14.8 |
| C | HUMIRA ® | 13 | 0.5 | −96.2% | −12.5 |
| C | STELARA ® | 13 | 12 | −7.7% | −1 |
| D | HUMIRA ® | 7.5 | 15 | 100.0% | 7.5 |
| D | STELARA ® | 7.5 | 0.8 | −89.3% | −6.7 |
| E | HUMIRA ® | 25 | 12 | −52.0% | −13 |
| E | TREMFYA ® | 8 | 4.3 | −46.3% | −3.7 |
| E | COSENTYX ® | 8 | 1.2 | −85.0% | −6.8 |
| F | HUMIRA ® | 4.1 | 0.8 | −80.5% | −3.3 |
| F | SIMPONI ® | 4.1 | 0.4 | −90.2% | −3.7 |
| G | TALTZ ® | 31.2 | 0 | −100.0% | −31.2 |
| G | STELARA ® | 56.5 | 22.4 | −60.4% | −34.1 |
| G | COSENTYX ® | 22.4 | 2.4 | −89.3% | −20 |
| H | STELARA ® | 11.4 | 3.5 | −69.3% | −7.9 |
| H | HUMIRA ® | 15.5 | 11.4 | −26.5% | −4.1 |
| H | COSENTYX ® | 15.3 | 1 | −93.5% | −14.3 |
| I | HUMIRA ® | 3.8 | 2 | −47.4% | −1.8 |
| I | COSENTYX ® | 2 | 0 | −100.0% | −2 |
| J | STELARA ® | 15 | 3.7 | −75.3% | −11.3 |
| J | TALTZ ® | 11.7 | 0 | −100.0% | −11.7 |
| A | TALTZ ® | 23.32 | 24.41 | 4.7% | 1.09 |
| A | STELARA ® | 23.32 | 22.33 | −4.2% | −0.99 |
| B | HUMIRA ® | 13.44 | 24.8 | 84.5% | 11.36 |
| B | STELARA ® | 13.44 | 13.03 | −3.1% | −0.41 |
| C | HUMIRA ® | 3.81 | 4.14 | 8.7% | 0.33 |
| C | STELARA ® | 3.81 | 3.92 | 2.9% | 0.11 |
| D | HUMIRA ® | 14.38 | 16.28 | 13.2% | 1.9 |
| D | STELARA ® | 14.38 | 13.93 | −3.1% | −0.45 |
| E | HUMIRA ® | 7.61 | 8.18 | 7.5% | 0.57 |
| E | TREMFYA ® | 7.61 | 6.9 | −9.3% | −0.71 |
| E | COSENTYX ® | 7.61 | 6.19 | −18.7% | −1.42 |
| F | HUMIRA ® | 9.85 | 12.2 | 23.9% | 2.35 |
| F | SIMPONI ® | 9.85 | 12.35 | 25.4% | 2.5 |
| G | TALTZ ® | 8.53 | 6.97 | −18.3% | −1.56 |
| G | STELARA ® | 8.53 | 8.94 | 4.8% | 0.41 |
| G | COSENTYX ® | 8.53 | 7.38 | −13.5% | −1.15 |
| H | STELARA ® | 7.55 | 7.83 | 3.7% | 0.28 |
| H | HUMIRA ® | 7.55 | 10.54 | 39.6% | 2.99 |
| H | COSENTYX ® | 7.55 | 5.96 | −21.1% | −1.59 |
| I | HUMIRA ® | 22.83 | 26.82 | 17.5% | 3.99 |
| I | COSENTYX ® | 22.83 | 20.54 | −10% | −2.29 |
| J | STELARA ® | 27.22 | 27.48 | 1% | 0.26 |
| J | TALTZ ® | 27.22 | 27.75 | 1.9% | 0.53 |

TABLE 4
STATISTICAL ANALYSIS

| | The reduction | The reduction |
|---|---|---|
| | ratio of PASI | difference of |
| | and IFN-γ/IL-4 | PASI and IFN-γ/IL-4 |
| Spearman's rho | | |
| r | 0.53 | 0.44 |
| P-value | 0.01 | 0.036 |

For example, for patient G, compared with before administration of TALTZ® or STELARA® or COSENTYX®, the rate of change in PASI decreased by 100% and 60.4% and 89.3%, respectively, after treatment. The difference of PASI decreased by 31.2 and 34.1 and 20, respectively; at the same time, the change rate of the ratio of interferon-γ/interleukin-4 decreased by 18.3% and −4.8% and 13.5%, respectively, while the ratio of interferon-γ/interleukin-4 decreased by 18.3%, −4.8% and 13.5%, respectively. The difference in the ratio of interferon-γ/interleukin-4 is decreased by 1.56 and −0.41 and 1.15, respectively. Accordingly, the present example further analyzes the relationship between PASI and the ratio of interferon-γ/interleukin-4 before and after treatment, and is summarized in Table 4. Based on the results in Table 4 above, it can be seen that the reduction ratio of PASI before and after treatment with different biological agents is significantly correlated with the reduction ratio of the patient's interferon-γ/interleukin-4 ratio, and the difference between the PASI reduction and the patient's interferon-γ/interleukin-4 ratio is significantly correlated. Accordingly, the change in the expression of the ratio of interferon-γ/interleukin-4 before and after treatment (whether it is the decreasing ratio or the decreasing difference) can be used as a medication index for psoriasis patients.

TABLE 5
PASI and IFN-γ/IL-9 ratio

| | Experienced | | | PASI | PASI |
|---|---|---|---|---|---|
| Pt | Biologics | PASI (Before) | PASI (After) | reduction ratio | reduction difference |
| A | TALTZ ® | 15 | 1.2 | −92.0% | −13.8 |
| A | STELARA ® | 16 | 0 | −100.0% | −16 |
| B | HUMIRA ® | 15 | 16 | 6.7% | 1 |
| B | STELARA ® | 15 | 0.2 | −98.7% | −14.8 |
| C | HUMIRA ® | 13 | 0.5 | −96.2% | −12.5 |
| C | STELARA ® | 13 | 12 | −7.7% | −1 |
| D | HUMIRA ® | 7.5 | 15 | 100.0% | 7.5 |
| D | STELARA ® | 7.5 | 0.8 | −89.3% | −6.7 |
| E | HUMIRA ® | 25 | 12 | −52.0% | −13 |
| E | TREMFYA ® | 8 | 4.3 | −46.3% | −3.7 |
| E | COSENTYX ® | 8 | 1.2 | −85.0% | −6.8 |
| F | HUMIRA ® | 4.1 | 0.8 | −80.5% | −3.3 |
| F | SIMPONI ® | 4.1 | 0.4 | −90.2% | −3.7 |
| G | TALTZ ® | 31.2 | 0 | −100.0% | −31.2 |
| G | STELARA ® | 56.5 | 22.4 | −60.4% | −34.1 |
| G | COSENTYX ® | 22.4 | 2.4 | −89.3% | −20 |
| H | STELARA ® | 11.4 | 3.5 | −69.3% | −7.9 |
| H | HUMIRA ® | 15.5 | 11.4 | −26.5% | −4.1 |
| H | COSENTYX ® | 15.3 | 1 | −93.5% | −14.3 |
| I | HUMIRA ® | 3.8 | 2 | −47.4% | −1.8 |
| I | COSENTYX ® | 2 | 0 | −100.0% | −2 |
| J | STELARA ® | 15 | 3.7 | −75.3% | −11.3 |
| J | TALTZ ® | 11.7 | 0 | −100.0% | −11.7 |
| K | HUMIRA ® | 3.2 | 2.6 | −18.8% | −0.6 |
| K | STELARA ® | 3.2 | 0 | −100.0% | −3.2 |
| A | TALTZ ® | 2.76 | 1.123 | −59.3% | −1.637 |
| A | STELARA ® | 2.76 | 1.307 | −52.6% | −1.453 |
| B | HUMIRA ® | 2.417 | 3.241 | 34.1% | 0.824 |
| B | STELARA ® | 2.417 | 2.198 | −9.1% | −0.219 |
| C | HUMIRA ® | 0.959 | 0.724 | −24.5% | −0.235 |
| C | STELARA ® | 0.959 | 0.829 | −13.6% | −0.13 |
| D | HUMIRA ® | 1.6 | 1.632 | 2% | 0.032 |
| D | STELARA ® | 1.6 | 1.506 | −5.9% | −0.094 |
| E | HUMIRA ® | 0.92 | 0.749 | −18.6% | −0.171 |
| E | TREMFYA ® | 0.92 | 0.836 | −9.1% | −0.084 |
| E | COSENTYX ® | 0.92 | 0.839 | −8.8% | −0.081 |
| F | HUMIRA ® | 2.273 | 2.55 | 12.2% | 0.277 |
| F | SIMPONI ® | 2.273 | 2.45 | 7.8% | 0.177 |
| G | TALTZ ® | 2.04 | 1.609 | −21.1% | −0.431 |
| G | STELARA ® | 2.04 | 2.257 | 10.6% | 0.217 |
| G | COSENTYX ® | 2.04 | 1.805 | −11.5% | −0.235 |
| H | STELARA ® | 1.893 | 1.951 | 3.1% | 0.058 |
| H | HUMIRA ® | 1.893 | 2.2 | 16.2% | 0.307 |
| H | COSENTYX ® | 1.893 | 1.474 | −22.1% | −0.419 |
| I | HUMIRA ® | 2.653 | 3.13 | 18% | 0.477 |
| I | COSENTYX ® | 2.653 | 2.75 | 3.7% | 0.097 |
| J | STELARA ® | 2.76 | 1.307 | −59.3% | −1.637 |
| J | TALTZ ® | 2.76 | 1.123 | −52.6% | −1.453 |
| K | HUMIRA ® | 0.623 | 0.656 | 5.3% | 0.033 |
| K | STELARA ® | 0.623 | 0.537 | −13.8% | −0.086 |

TABLE 6
STATISTICAL ANALYSIS

| | The reduction ratio of PASI | The reduction difference of |
|---|---|---|
| | and IFN-γ/IL-9 | PASI and IFN-γ/IL-9 |
| Spearman's rho | | |
| r | 0.45 | 0.6 |
| P-value | 0.025* | 0.005** |
| | Significant difference | Significant difference |

Based on above-mentioned table 5 result as can be known, for a specific psoriasis patient, cast different biological preparations for treatment, can cause the change ratio or difference of its PASI to produce different degrees of decline, meanwhile, also find interference The change ratio or difference of the ratio of IL-γ/IL-9 will decrease in different degrees. For example, for patient G, compared with before administration of TALTZ®? or STELARA®? or COSENTYX®?, the rate of change in PASI decreased by 100% and 60.4% and 89.3%, respectively, after treatment. The difference of PASI decreased by 31.2 and 34.1 and 20, respectively; at the same time, the change rate of its IFN-γ/IL-9 ratio decreased by 21.1% and −10.6% and 11.5%, respectively. The difference in the ratio of IFN-γ/IL-9 decreased by 0.431 and −0.217 and 0.235 respectively. Accordingly, this example further analyzes the relationship between PASI and the ratio of IFN-γ/IL-9 before and after treatment, and is summarized in Table 6. Based on the results in Table 6 above, it can be seen that the reduction ratio of PASI before and after treatment with different biological agents is significantly correlated with the reduction ratio of the patient's IFN-γ/IL-9 ratio, and the difference between the PASI reduction and the patient's IFN-γ/IL-9 ratio is significantly correlated. Accordingly, the change in the expression of the ratio of interferon-γ/interleukin-9 before and after treatment (whether it is the decreasing ratio or the decreasing difference) can be used as a medication index for psoriasis patients.

From the results of the screening method of the present invention for patient G, it is found that the peripheral blood mononuclear cells of the patient have a better response to TALTZ® (the obtained interferon-γ/interleukin-4 ratio is the smallest: 6.97 or the reference interference The ratio of interleukin-gamma/interleukin-9 is the smallest: 1.609). Accordingly, the follow-up treatment is switched to TALTZ®, and after 6 months of treatment, it is found that the patient's condition showed significant improvement, and the patient's PASI also changed from the original TALTZ® 31.2 before treatment progressed to 0 after treatment.

Based on the above results, the screening method of the present invention evaluates the variations in the expression of inflammatory cytokines secreted by cells in vitro (the variation in the ratio of the expression of IFN-γ/the expression of IL-4; represented as the reduction ratio or reduction difference) or the variations in the ratio of IFN-γ expression/IL-9 expression; represented as the reduction ratio or reduction difference), which can be used as the medication basis for the individual providing the cells to improving the drug efficacy.

In the above-mentioned examples, the method of screening a therapeutic drug from a plurality of candidate drugs can reduce the ratio of the expression of IFN-γ to the expression of IL-4 for the therapeutic drug. In one embodiment, the screened therapeutic drug is selected from top 40% of the candidate drugs ranked in order from having a smallest ratio to a largest ratio of the IFN-γ expression level to the IL-4 expression level, or selected from top 40% of the candidate drugs ranked in order from having a smallest ratio to a largest ratio of the IFN-γ expression level to the IL-9 expression level. In one embodiment, the screened therapeutic drug has a minimum ratio of the IFN-γ expression level to the IL-4 expression level or a minimum ratio of the IFN-γ expression level to the IL-9 expression level. Because the judging method is the relationship between the ratio of IFN-γ and interleukin, the smaller the numerator or the larger the denominator, the smaller the ratio. Therefore, in another embodiment, a candidate drug can be selected as a therapeutic drug if the candidate drug can cause a minimum expression level of IFN-γ, a maximum expression level of IL-4, or a maximum expression level of IL-9.

### Embodiment 3, IFN-γ and IL-13

The peripheral blood mononuclear cells are activated with a stimulus, so that the cells produce IFN-γ and IL-13. The expression levels of IFN-γ and IL-13 are determined, and a therapeutic drug is selected from the plurality of candidate drugs. The variation in the expression levels of IFN-γ and IL-13 in step (b) (that is, before administration of a specific drug candidate), and the expressed amounts of IFN-γ and IL-13 in step (d) (ie, after administration of a specific drug candidate). The relevant calculation formula is as follows, wherein formula (V) is the formula for calculating the rate of change of the expression of interferon-γ, and formula (VI) is the ratio of the expression of IFN-γ to the expression of IL-13. The formula for the rate of change.

$$Δ IFN - γ = \frac{step ( d ) IFN - γ - step ( b ) IFN - γ}{step ( b ) IFN - γ} \qquad formula ( V )$$

$$\begin{matrix}\frac{\begin{matrix}( IFN - γ / IL - 13 w / S . pyogenes \& biologics ) - \\ ( IFN - γ / IL - 13 w / S . pyogenes only )\end{matrix}}{( IFN - γ / IL - 13 w / S . pyogenes only )} \\ ( \frac{step ( d ) ( IFN - γ / IL - 13 - step ( b ) IFN - γ / IL - 13 )}{step ( b ) IFN - γ / IL - 13} )\end{matrix} \qquad formula ( VI )$$

### Embodiment 3.1 IFN-γ

TABLE 7
PASI and IFN-γ

| | Experienced | PASI | PASI | PASI reduction | PASI reduction |
|---|---|---|---|---|---|
| Pt | Biologies | (Before) | (After) | ratio | difference |
| A | HUMIRA ® | 3.2 | 2.6 | −18.8% | −0.6 |
| A | STELARA ® | 3.2 | 0 | −100.0% | −3.2 |
| B | HUMIRA ® | 15 | 16 | 6.7% | 1 |
| B | STELARA ® | 15 | 0.2 | −98.7% | −14.8 |
| C | HUMIRA ® | 13 | 0.5 | −96.2% | −12.5 |
| C | STELARA ® | 13 | 12 | −7.7% | −1 |
| D | HUMIRA ® | 7.5 | 15 | 100.0% | 7.5 |
| D | STELARA ® | 7.5 | 0.8 | −89.3% | −6.7 |
| E | HUMIRA ® | 25 | 0.4 | −98.4% | −24.6 |
| E | TREMFYA ® | 8 | 4.3 | −46.3% | −3.7 |
| E | COSENTYX ® | 8 | 1.2 | −85.0% | −6.8 |
| A | HUMIRA ® | 13.72 | 12.43 | −9.4% | −1.29 |
| A | STELARA ® | 13.72 | 11.97 | −12.8% | −1.75 |
| B | HUMIRA ® | 74.46 | 88.28 | 18.6% | 13.82 |
| B | STELARA ® | 74.46 | 71.02 | −4.6% | −3.44 |
| C | HUMIRA ® | 22.9 | 19.71 | −13.9% | −3.19 |
| C | STELARA ® | 22.9 | 21.97 | −4.1% | −0.93 |
| D | HUMIRA ® | 200.39 | 222.11 | 10.8% | 21.72 |
| D | STELARA ® | 200.39 | 201.66 | 0.6% | 1.27 |
| E | HUMIRA ® | 92.6 | 76.99 | −16.9% | −15.61 |
| E | TREMFYA ® | 92.6 | 85.17 | −8.0% | −7.43 |
| E | COSENTYX ® | 92.6 | 80.12 | −13.5% | −12.48 |

TABLE 8
STATISTICAL ANALYSIS

| | PASI reduction | PASI reduction |
|---|---|---|
| | ratio and IFN-γ | ratio and IFN-γ |
| | reduction ratio | reduction difference |
| Spearman's rho | | |
| r | 0.6364 | −0.6182 |
| 95% confidence | 0.03841 to 0.8987 | −0.8928 to −0.008442 |
| interval | | |
| P-value | 0.0402 | 0.0478 |
| P-value | * | * |
| | Significant difference | Significant difference |
| | PASI reduction | PASI reduction |
| | difference and | difference and |
| | IFN-γ reduction ratio | IFN-γ reduction difference |
| Spearman's rho | | |
| r | −0.6273 | 0.7818 |
| 95% confidence | −0.8958 to −0.02329 | 0.3243 to 0.9429 |
| interval | | |
| P-value | 0.044 | 0.0064 |
| P-value | * | ** |
| | Significant difference | Significant difference |

Based on the above-mentioned table 7 results, it can be known that for a psoriasis patient, administration of different biological agents for treatment can cause the change ratio or difference of its PASI to produce different degrees of decline (that is, the patient's psoriasis situation obtained different degrees of improvement), at the same time, it is also found that the change ratio or difference of the amount of IFN-γ expression would decrease to different degrees. For example, in patient A, compared with before the administration of HUMIRA® or STELARA®, the rate of change in PASI decreased by 18.8% and 100.0%, and the difference in PASI decreased, respectively. 0.6 and 3.2; at the same time, the rate of change in the amount of interferon-γ expression decreased by 9.4% and 12.8%, respectively, and the difference in the amount of interferon-γ expression decreased by 1.29 and 1.75 μg/ml, respectively, and so on. Accordingly, this example further analyzes the relationship between PASI and IFN-γ changes before and after treatment by statistical analysis, which is summarized in Table 8. Based on the results in Table 8, it can be seen that the reduction ratio of PASI before and after treatment with different biological agents is significantly correlated with the reduction ratio or the difference of the patient's IFN-γ. Both the ratio of declines or the difference in declines in γ are significantly correlated. Accordingly, the change in the expression of IFN-γ before and after treatment (whether it is the decreasing ratio or the decreasing difference) can be used as a medication index for psoriasis patients.

### Embodiment 3.2 IFN-γ and IL-13

TABLE 9
PASI and the ratio IFN-γ/ IL-13

| | Experienced | PASI | PASI | PASI reduction | PASI reduction |
|---|---|---|---|---|---|
| Pt | Biologics | (Before) | (After) | ratio | difference |
| A | TALTZ ® | 15 | 1.2 | −92.0% | −13.8 |
| A | STELARA ® | 16 | 0 | −100.0% | −16 |
| B | HUMIRA ® | 15 | 16 | 6.7% | 1 |
| B | STELARA ® | 15 | 0.2 | −98.7% | −14.8 |
| C | HUMIRA ® | 13 | 0.5 | −96.2% | −12.5 |
| C | STELARA ® | 13 | 12 | −7.7% | −1 |
| D | HUMIRA ® | 7.5 | 15 | 100.0% | 7.5 |
| D | STELARA ® | 7.5 | 0.8 | −89.3% | −6.7 |
| E | HUMIRA ® | 25 | 12 | −52.0% | −13 |
| E | TREMFYA ® | 8 | 4.3 | −46.3% | −3.7 |
| E | COSENTYX ® | 8 | 1.2 | −85.0% | −6.8 |
| A | TALTZ ® | 127.3 | 132.63 | 4.2% | 5.33 |
| A | STELARA ® | 127.3 | 119.85 | −5.9% | −7.45 |
| B | HUMIRA ® | 60.05 | 108.99 | 81.5% | 48.94 |
| B | STELARA ® | 60.05 | 57.27 | −4.6% | −2.78 |
| C | HUMIRA ® | 45.8 | 33.4 | −27.1% | −12.4 |
| C | STELARA ® | 45.8 | 43.82 | −4.3% | −1.98 |
| D | HUMIRA ® | 38.6 | 51.53 | 33.5% | 12.93 |
| D | STELARA ® | 38.6 | 38.19 | −1.1% | −0.41 |
| E | HUMIRA ® | 141.95 | 151.67 | 6.8% | 9.72 |
| E | TREMFYA ® | 141.95 | 155.73 | 9.7% | 13.78 |
| E | COSENTYX ® | 141.95 | 128.9 | −9.2% | −13.05 |

TABLE 10
STATISTICAL ANALYSIS

| | PASI reduction ratio and | PASI reduction ratio and |
|---|---|---|
| | IFN-γ/IL-13 reduction | IFN-γ/IL-13 reduction |
| | ratio | difference |
| Spearman's rho | | |
| r | 0.7364 | −0.6818 |
| 95% confidence interval | 0.2250 to 0.9297 | −0.9131 to −0.1183 |
| P-value | 0.0128 | 0.0251 |
| | Significant difference | Significant difference |
| | PASI reduction difference and | PASI reduction difference and |
| | IFN-y/IL-13 reduction ratio | IFN-y/IL-13 reduction |
| | | difference |
| Spearman's rho | | |
| r | −0.5818 | 0.5455 |
| 95% confidence interval | −0.8807 to 0.04837 | −0.1014 to 0.8681 |
| P-value | 0.0656 | 0.0876 |
| P-value | Not significant | Not significant |

Based on the results of above-mentioned table 9, it can be known that for a psoriasis patient, throwing different biological agents for treatment can cause the change ratio of its PASI or the difference to produce different degrees of decline, at the same time, it is also found that interference The change ratio or difference of the ratio of IL-γ/IL-13 will decrease in different degrees. For example, for patient C, compared with before administration of HUMIRA® or STELARA®, the rate of change in PASI decreased by 96.2% and 7.7%, respectively, and the difference in PASI decreased after treatment. 12.5 and 1; at the same time, the change ratio of its IL-γ/IL-13 ratio decreased by 27.1% and 4.3%, respectively, and the difference of its IL-γ/IL-13 ratio decreased by 12.4 and 1.98, and so on. Based on the results in Table 14 above, it can be seen that the reduction ratio of PASI before and after treatment with different biological agents is significantly related to the reduction ratio or the difference of the ratio of IL-γ/IL-13 of the patient, but the difference of PASI reduction is There is no significant correlation between the ratio of declines or the difference in declines in the patient's IL-γ/IL-13 ratio. Accordingly, the change in the expression of the ratio of IL-γ/IL-13 before and after treatment (whether it is the decreasing ratio or the decreasing difference) can be used as a medication index for psoriasis patients.

Utilize the ratio of IFN-γ/IL-13 change before and after treatment can be used as the index of psoriasis patient medication, therefore, the present embodiment then evaluates the in vitro first screening method. Initially use the cell screening platform of the present invention in vitro to screen out a drug suitable for the patient from which the cell is derived, and then administer the screened drug to the patient.

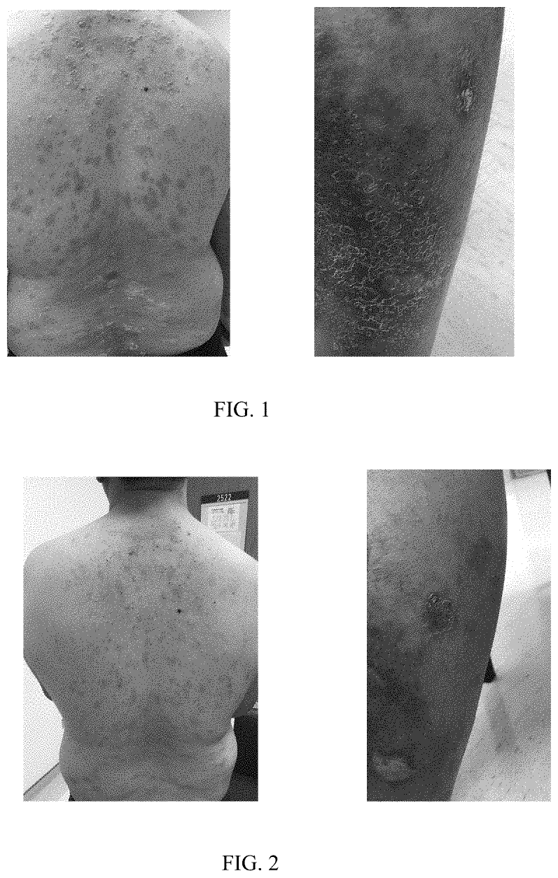

Patient C in Table 7 and Table 9 is initially treated with STELARA® for six months after treatment, no significant improvement is found. The psoriasis lesions are red, and the lesions are thick by visual inspection, and there is a lot of desquamation at close range (see, FIG. 1). In order to seek the best therapeutic drug for the patient, the peripheral blood mononuclear cells of the patient are used to carry out the drug screening method of the present invention. From the results of the screening method of the present invention, it is found that the peripheral blood mononuclear cells of the patient have a better response to HUMIRA® (IFN-γ/IL-13 obtained after administration) The ratio of IFN-γ/IL-13 is HUMIRA®: 33.4, and STELARA®: 43.82). Accordingly, the follow-up treatment is switched to HUMIRA®, and after 6 months of treatment, it is found that the patient's condition showed significant improvement, with psoriatic lesions. The redness subsided, the thickness of the lesion did not increase, and the desquamation is reduced (see, FIG. 2), and the patient's PASI also improved from 13 before HUMIRA® treatment to 0.5 after HUMIRA® treatment.

Based on the results, the screening method by evaluating the changes in the expression levels of cells secreting inflammatory cytokines in vitro (for example, the rate of reduction in the expression of IFN-γ, or the reduction difference in expression level of IFN-γ/IL-13 expression ratio), as the basis for the drug use of the individual from which the cells are derived, can indeed achieve the purpose of significantly improving the efficacy of the drug, and has the benefit of requesting invention patent protection. In other embodiments, if a candidate drug can minimize the expression of interferon-γ or maximize the expression of interleukin-13, ratio of IFN-γ/IL-13 significantly decreased, the candidate drug can also be selected as a therapeutic drug.

Drug-Induced Psoriasis Arthritis

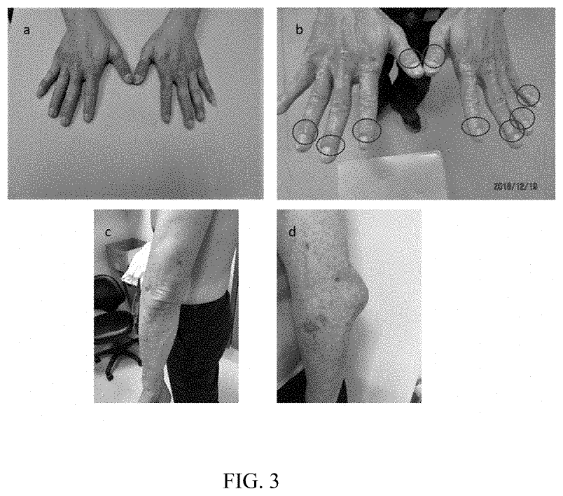

In psoriasis, there are several researches point out benefits for preventing occurrence of psoriatic arthritis (PsA). However, there are rare cases presenting paradoxical arthritis after receiving biologics which means psoriatic arthritis outbreak while receiving drug treatment on PsA patients or purely psoriasis patients without PsA. Theses biologics included anti-TNF-a, anti-IL17, anti-IL-12/IL-23, and anti-IL-23. FIG. 3 shows clinical pictures of patients with drug-induced paradoxical psoriatic arthritis. The mechanism about paradoxical arthritis is still unclear. Twenty-two psoriasis patients with or without psoriatic arthritis are enrolled in this study. All participants provided written informed consent. The protocols and all research involving human participants are approved by the Institutional Review Board of Taichung Veterans General Hospital (TCVGH-CE16265B; TCVGH-CE20043B).

Table 11 below lists a laboratory profile of 2 paradoxial PsA and non-paradoxial PsA Psoriasis Patients on IL-6, IP-10, interferon-γ, GM-CSF, PDGF, MCP-1, MIP-1α, RANTES, and VEGF (n=22).

| Pt | | | | IFN-r | Hu IP-10 (MCP-1 | | MIP-1α | MIP-1β (1 PDGF | | RANTES | VEGF | IL-6 |
|---|---|---|---|---|---|---|---|---|---|---|---|---|
| P1 | S.Coccus (S.C) | | | 47.44 | 54.91 | 1137.46 | 1690.81 | 1760.66 | 269.16 | 353.15 | 327.98 | 1355.94 |
| P1 | S.Coccus (S.C) + Cosentyx | | | 39.49 | 37.91 | 1245.98 | 2146.98 | 2419.11 | 255.74 | 1565.77 | 311.7 | 2122.65 |
| P1. | (Cosentyx-S.Coccus)/S.Coccus | P1 | | −16.8% | −31.0% | 9.5% | 27.0% | 37.4% | −5.0% | 343.4% | −5.0% | 56.5% |
| P2 | S.Coccus (S.C) | | | 912.32 | 29622.5 | 4590.08 | 1810.96 | | 700.46 | 1095.43 | 570.43 | 9997.26 |
| P2 | S.Coccus (S.C) + brodalumab | | | 626 | 30864.1 | 5577.46 | 1759.99 | | 659.83 | 2175.75 | 407.15 | 9138 14 |
| P2. | (lumicef-S.Coccus)/S.Coccus | P2 | | −31.4% | 4.2% | 21.5% | −2.8% | | −5.8% | 98.6% | −28.6% | −8.6% |
| 1. | S.Coccus | | | 13.72 | 29.07 | 22.48 | 612.95 | 552.4 | 90.57 | 456.82 | 321.43 | 269.08 |
| 1. | Humira | | | 12.43 | 29.07 | 22.52 | 578.63 | 565.3 | 83.43 | 406.53 | 362.62 | 253.47 |
| 1. | Stelera | | | 11.97 | 29.79 | 19.46 | 623.99 | 521.94 | 89.78 | 431.08 | 351.95 | 233.74 |
| 1. | (Humira-Cstrep)/Cstrep | | 1 | −9.4% | 0.0% | 0.2% | −5.6% | 2.3% | −7.9% | −11.0% | 12.8% | 5.8% |
| 1. | (Stelera-Cstrep)/Cstrep | | 1 | −12.8% | 2.5% | −13.4% | 1.8% | −5.5% | −0.9% | −5.6% | 9.5% | −13.1% |
| 2. | S.Coccus | | | 22.9 | 43.38 | 43.74 | 372 | 890.65 | 142.31 | 609.94 | 484.97 | 468.01 |
| 2. | Humira | | | 19.71 | 40.36 | 43.11 | 703.67 | 915.7 | 126.32 | 666.78 | 503.61 | 425.03 |
| 2. | Stelera | | | 21.91 | 44.56 | 37.31 | 613.84 | 997.14 | 135.48 | 594.96 | 517.32 | 455.88 |
| 2. | (Humira-strep)/strep | | 2 | −13.9% | −7.0% | −1.4% | 4.7% | 2.8% | −11.2% | 9.3% | 3.8% | −9.2% |
| 2. | (Stelera-strep)/strep | | 2 | −4.3% | 2.7% | −14.7% | −8.7% | 12.0% | −4.8% | −2.5% | 6.7% | −2.6% |
| 3. | S.Coccus | | | 74.46 | 55.12 | 1671.89 | 1235.33 | | 96.09 | 198.59 | 1241.04 | 1992 34 |
| 3. | Humira | | | 88.26 | 104.2 | 1563.51 | 1116.58 | | 76.63 | 279.32 | 819.09 | 2134.34 |
| 3. | Stelera | | | 71.02 | 53.78 | 1662.14 | 1055.11 | | 94.52 | 233.98 | 686.24 | 207952 |
| 3. | (Humira-strep)/strep | | 3 | 18.6% | 89.0% | −6.5% | −9.6% | | −20.3% | 40.7% | −34.0% | 7.1% |
| 3. | (Stelera-strep)/strep | | 3 | −4.6% | −2.4% | −0.6% | −14.6% | | −1.6% | 17.8% | −44.7% | 4.4% |
| 4. | S.Coccus (S.C) | | | 107.72 | 153.1 | 1862.21 | 233.92 | | 203.03 | 1806.47 | 504 | 8438.26 |
| 4. | S.Coccus (S.C) + Simponi | | | 107.68 | 83.84 | 1925.28 | 229.23 | | 168.86 | 1490.32 | 447 | 8183.05 |
| 4. | S.Coccus (S.C) + Humira | | | 105.21 | 72.56 | 1874.86 | 233.72 | | 170.09 | 1510.73 | 401.5 | 8308.38 |
| 4. | (Simponi-S.Coccus)/S.Coccus | | 4 | 0.0% | −45.2% | 3.4% | −2.0% | | −16.8% | −17.5% | −11.3% | −3.0% |
| 4. | (Humira-S.Coccus)/S.Coccus | | 4 | −2.3% | −52.6% | 0.7% | −0.1% | | −16.2% | −16.4% | −20.3% | −1.5% |
| 5. | S.Coccus (S.C) | | | 85.68 | 61.6 | 1765.35 | 225.93 | 225.93 | 137.07 | 850.16 | 541.68 | 7877.98 |
| 5. | S.Coccus (S.C) + Stelera | | | 89.51 | 63.4 | 1866.09 | 233.75 | 233.75 | 133.18 | 568.75 | 511.92 | 8619.83 |
| 5. | S.Coccus (S.C) + Humira | | | 102.91 | 68.38 | 1870.76 | 226.72 | 226.72 | 165.15 | 720 | 492.84 | 8179.9 |
| 5. | S.Coccus (S.C) + Cosentyx | | | 66 92 | 61.47 | 1828.06 | 228.91 | 228.91 | 183.53 | 619.31 | 529.31 | 8335.37 |
| 5. | (Stelera-S.Coccus)/S.Coccus | | 5 | 4.5% | 2.9% | 5.7% | 3.5% | 3.5% | −2.0% | −33.1% | −5.5% | 9.4% |
| 5. | (Humira-S.Coccus)/S.Coccus | | 5 | 20.1% | 11.0% | 6.0% | 0.3% | 0.3% | −16.2% | −15.3% | −9.0% | 3.8% |
| 5. | (Cosentyx-S.Coccus)/S.Coccus | | 5 | −21.9% | −0.2% | 3.6% | 1.3% | 1.3% | −6.9% | −27.2% | −2.3% | 5.8% |
| 6. | S.Coccus (S.C) | | | 100.13 | 56.56 | 1859.3 | 229.68 | 20848 | 218.66 | 1473.91 | 510.52 | 8035.57 |
| 6. | S.Coccus (S.C) + Taltz | | | 76 76 | 74.62 | 1838.34 | 224.09 | 17333.8 | 203.92 | 1024.4 | 485.69 | 8100.99 |
| 6. | S.Coccus (S.C) + Stelera | | | 102.31 | 62.44 | 1839.49 | 231.98 | 18810.8 | 205.11 | 885.03 | 486.41 | 7850.62 |
| 6. | S.Coccus (S.C) + Humira | | | 105.29 | 96.05 | 1829 | 224.43 | 17105.5 | 201.54 | 1429.38 | 509.82 | 7701.18 |
| 6. | S.Coccus (S.C) + Cosentyx | | | 84.74 | 49.93 | 1850.23 | 224.29 | 17655.7 | 206 | 1083.71 | 528.62 | 7875.04 |
| 6. | (Taltz-S.Coccus)/S.Coccus | | 6 | −23.3% | 31.9% | −1.1% | −2.4% | −16.9% | −6.7% | −30.5% | 4.9% | 0.8% |
| 6. | (Stelera-S.Coccus)/S.Coccus | | 6 | 2.2% | 10.4% | −1.1% | 1.0% | −9.8% | −6.2% | −40.0% | 4.7% | −2.3% |
| 6. | (Humira-S.Coccus)/S.Coccus | | 6 | 5.2% | 69.8% | −1.6% | −2.3% | −18.0% | −7.8% | −3.0% | −0.1% | 4.2% |
| 6. | (Cosentyx-S.Coccus)/S.Coccus | | 6 | −15.4% | −11.7% | −0.5% | −2.3% | −15.3% | −5.8% | −26.5% | 3.5% | −2.0% |
| 7. | S.Coccus (S.C) | | | 217.19 | 26.95 | 1672.21 | 379.13 | | 203 | 3485.13 | 248.92 | 7053.04 |
| 7. | S.Coccus (S.C) + Stelera | | | 232.06 | 30.84 | 1624.31 | 378.59 | | 171.43 | 3481.16 | 229.24 | 7266.03 |
| 7. | S.Coccus (S.C) + Humira | | | 218.14 | 30.5 | 1664.34 | 391.08 | | 199.23 | 3371.83 | 257.8 | 7056.76 |
| 7. | S.Coccus (S.C) + tremfya | | | 208.68 | 28.64 | 1480.86 | 393.29 | | 195.46 | 3141.23 | 265.75 | 6969.67 |
| 7. | S.Coccus (S.C) + Cosentyx | | | 228.43 | 29.58 | 1326.05 | 392.88 | | 212.89 | 3625.9 | 265.22 | 6876.25 |
| 7. | (Stelera-S.Coccus)/S.Coccus | | 7 | 6.8% | 14.4% | −13.2% | −0.1% | | −15.6% | −0.1% | −7.9% | 3.0% |
| 7. | (Humira-S.Coccus)/S.Coccus | | 7 | 0.4% | 13.2% | −11.1% | 3.2% | | −1.9% | −3.3% | 3.6% | 0.1% |
| 7. | (tremfya-S.Coccus)/S.Coccus | | 7 | −3.9% | 6.3% | −20.9% | 3.7% | | −3.7% | −9.9% | 6.8% | −1.2% |
| 7. | (Cosentyx-S.Coccus)/S.Coccus | | 7 | 5.2% | 9.8% | −29.2% | 3.6% | | 4.9% | 4.0% | 6.5% | −2.5% |
| 8. | S.Coccus (S.C) | | | 273.76 | 957.47 | 2397.74 | 370.22 | | 441.02 | 3336.7 | 256.73 | 7162.94 |
| 8. | S.Coccus (S.C) + Taltz | | | 297.09 | 1072.98 | 2071.79 | 376.08 | | 477.47 | 3032.26 | 265.22 | 697186 |
| 8. | S.Coccus (S.C) + Stelera | | | 268.46 | 841.29 | 2342.12 | 391.37 | | 470.68 | 2867.91 | 257.8 | 7225.77 |
| 8. | (Taltz-S.Coccus)/S.Coccus | | 8 | 8.5% | 12.1% | −13.6% | 1.6% | | 8.3% | −9.1% | 3.3% | −2.7% |
| 8. | (Stelera-S.Coccus)/S.Coccus | | 8 | −1.9% | − 12.1% | −2.3% | 5.7% | | 6.7% | −14.0% | 0.4% | 0.9% |
| 9. | Streptococcus | | | 157.8 | 24.18 | 1947.67 | 421.8 | | 239.8 | 5431.76 | 191.61 | 6091.18 |
| 9. | Streptococcus + Cosentyx | | | 133.14 | 26.21 | 2180.51 | 467.25 | | 270.39 | 5405.58 | 203.56 | 7929.81 |
| 9. | (Cosentyx-Streptococcus)/Streptoc | | 9 | −15.6% | 8.4% | 12.0% | 10.8% | | 12.8% | −0.5% | 6.2% | 30.2% |
| 10. | S.Coccus (S.C) | | | 244.94 | 41.77 | 1637.36 | 379.79 | | 138.23 | 2716.11 | 230.93 | 7027.8 |
| 10. | S.Coccus (S.C) + Humira | | | 269.64 | 69.71 | 1926.28 | 389.11 | | 141.09 | 3231.73 | 251 89 | 7178.95 |
| 10. | S.Coccus (S.C) + Cosentyx | | | 269.05 | 44.2 | 1785.35 | 405.96 | | 162 | 2994.89 | 281.82 | 7757.38 |
| 10. | (Humira-S.Coccus)/S.Coccus | | 10 | 10.1% | 66.9% | 17.6% | 2.5% | | 2.1% | 19.0% | 9.1% | 2.2% |
| 10. | (Cosentyx-S.Coccus)/S.Coccus | | 10 | 9.8% | 5.8% | 9.0% | 6.9% | | 17.2% | 10.3% | 220% | 10.4% |
| 11. | S.pyogenes | | | 236.09 | 37.44 | 1631.91 | 390.99 | | 198.16 | 6092.31 | 250.54 | 6975.52 |
| 11. | S.pyogenes + Enbrel | | | 241.47 | 30.96 | 1812.78 | 386.78 | | 151.61 | 4697.63 | 226.68 | 6933.24 |
| 11. | (Enbrel-S.pyogenes)/S.pyogenes | | 11 | 2.3% | −17.3% | 11.1% | −1.1% | | −23.5% | −22.9% | −9.5% | −0.6% |
| 12. | S.Coccus (S.C) | | | 367.78 | 1238.93 | 3829.46 | 454.4 | | 298.73 | 4537.27 | 303.05 | 7636.72 |
| 12. | S.Coccus (S.C) + Taltz | | | 383.17 | 1223.7 | 4096.26 | 457.02 | | 295.93 | 4125.88 | 322.06 | 8360.4 |
| 12. | S.Coccus (S.C) + Stelara | | | 365.71 | 953.1 | 4086.31 | 448.7 | | 320.45 | 4639.91 | 313.35 | 8324.11 |
| 12. | (Taltz-S.Coccus)/S.Coccus | | 12 | 4.2% | −1.2% | 7.0% | 0.6% | | −0.9% | −9.1% | 6.3% | 9.5% |
| 12. | (Stelera-S.Coccus)/S.Coccus | | 12 | −0.6% | −23.1% | 6.7% | −1.3% | | 7.3% | 2.3% | 3.4% | 9.0% |
| 13. | S.Coccus (S.C) | | | 84.7 | 692.24 | 2683.3 | 1678.57 | | 583.22 | 2938.48 | 519.7 | 23203 |
| 13. | S.Coccus (S.C) + Humira | | | 69.96 | 306.01 | 3360.41 | 1659.65 | | 458.27 | 3022.08 | 432.02 | 24309.9 |
| 13. | S.Coccus (S.C) + Stelara | | | 80.04 | 410.2 | 3427.81 | 1800.57 | | 555.64 | 3114.53 | 496.57 | 25831.6 |
| 13. | (Humira-S.Coccus)/S.Coccus | | 13 | −17.4% | −55.8% | 25.2% | −1.1% | | −21.4% | 2.8% | −16.9% | 4.8% |
| 13. | (Stelera-S.Coccus)/S.Coccus | | 13 | −5.5% | −40.7% | 27.7% | 7.3% | | 4.7% | 6.0% | −4.5% | 11.3% |
| 14. | S.Coccus (S.C) | | | 66.37 | 2426.9 | 2032.6 | 9857.34 | 11476.7 | 408.2 | 3237.65 | 294.19 | 603.88 |
| 14. | Simponi | | | 66.72 | 4159.72 | 1463.88 | 4276.13 | 4194.61 | 417.86 | 2623.54 | 251.42 | 647.78 |
| 14. | Taltz | | | 63.72 | 1854.14 | 1806.97 | 15295.4 | 6159.65 | 346.46 | 2249.24 | 295.25 | 574.42 |
| 14. | Stelara | | | 61.96 | 2083.91 | 1354.84 | 10644.7 | 7636.11 | 422.69 | 2632.06 | 316.51 | 716.43 |
| 14. | (Simponi-S.Coccus)/S.Coccus | | 14 | 0.5% | 71.4% | −28.0% | −56.6% | 63.5% | 2.4% | −19.0% | −14.5% | 7.3% |
| 14. | (Taltz-S.Coccus)/S.Coccus | | 14 | −4.0% | −23.6% | −11.1% | 55.2% | 46.3% | −15.1% | −30.5% | 0.4% | 4.9% |
| 14. | Stelera-S.Coccus)/S.Coccus | | 14 | 6.6% | −14.1% | −33.3% | 8.0% | −33.5% | 3.5% | −18.7% | 7.6% | 18.6% |
| 15. | S.Coccus (S.C) | | | 54.73 | 147.53 | 1333.22 | 3009.58 | 2147.29 | 177.72 | 536.54 | 256.06 | 456.76 |
| 15. | Simponi | | | 49.66 | 114.8 | 777.67 | 667.29 | 1224.81 | 131.83 | 247.34 | 262.93 | 297.8 |
| 15. | Humira | | | 51.54 | 150.49 | 1296.09 | 1999.09 | 1856.42 | 151.72 | 549.19 | 249.66 | 529.11 |
| 15. | (Simponi-S.Coccus)/S.Coccus | | 15 | −9.3% | −22.2% | 41.7% | −77.8% | −43.0% | −25.8% | −53.9% | 2.7% | −34.8% |
| 15. | (Humira-S.Coccus)/S.Coccus | | 15 | −5.8% | 2.0% | −2.8% | −33.6% | −13.5% | −14.6% | 2.4% | −2.5% | 15.8% |
| 16. | S.Coccus (S.C) | | | 111.16 | 147.53 | 2657.21 | 45213.2 | 2089.33 | 156.69 | 188.05 | 256.64 | 3075.84 |
| 16. | Simponi | | | 55.51 | 107.23 | 2228.19 | 109677 | 2006.39 | 154.21 | 203.09 | 281.83 | 2696.67 |
| 16. | (Simponi-S.Coccus)/S.Coccus | | 16 | −50.1% | −27.3% | −16.1% | 142.6% | 4.0% | −1.6% | 80% | 9.8% | −12.3% |
| 17. | S.Coccus (S.C) | | | 50 | 173.22 | 2753.83 | 715888 | 3266.77 | 109.35 | 126.43 | 247.9 | 2985.91 |
| 17. | Taltz | | | 53.26 | 68.82 | 2538.78 | | 4431.27 | 116.86 | 123 35 | 252 58 | 3271 52 |
| 17. | Tremfya | | | 40.67 | 137.82 | 3511.38 | | 2625.4 | 91.78 | 98.09 | 251.42 | 2630.22 |
| 17. | (Taltz-S.Coccus)/S.Coccus | | 17 | −18.7% | −20.4% | 27.5% | −100.0% | −19.6% | −16.1% | −22.4% | 1.4% | −11.9% |
| 17. | (Tremfya-S.Coccus)/S.Coccus | | 17 | −18.7% | −20.4% | 27.5% | −100.0% | −19.6% | −16.1% | −22.4% | 1.4% | −11.9% |
| 18. | S.Coccus (S.C) | | | 194.87 | 59.77 | 2202 | 507.13 | | 176.39 | 272.3 | 320.98 | 7410 |
| 18. | S.Coccus (S.C) + Stelara | | | 134.85 | 45.92 | 489.93 | 367.59 | | 153.02 | 203.52 | 260.46 | 4805.17 |
| 18. | S.Coccus (S.C) + Taltz | | | 81.47 | 53.99 | 11482.8 | 445.74 | | 130.72 | 147.71 | 222.67 | 3860.73 |
| 18. | (Stelera-S.Coccus)/S.Coccus | | 18 | −30.8% | −23.2% | −77.8% | −27.5% | | −13.2% | −25.3% | −18.9% | −35.2% |
| 18. | (Taltz-S.Coccus)/S.Coccus | | 18 | −58.2% | − 9.7% | 421.5% | −12.1% | | −25.9% | −45.8% | −30.6% | −47.9% |
| 19. | S.pyogens | | | 247.16 | 30.16 | 6430.92 | 368.08 | | 104.31 | 1420.4 | 188.17 | 6907.54 |
| 19. | Humira | | | 262.11 | 79.83 | 7498.37 | 366.99 | | 168.11 | 1480.06 | 293.21 | 7427.64 |
| 19. | Cosentyx | | | 297.59 | 63.53 | 7922.35 | 466.84 | | 214.75 | 2539.13 | 357.88 | 9129.7 |
| 19. | (Humira-S.pyogens)/S.pyogens | | 19 | 6.0% | 164.7% | 16.6% | −0.3% | | 61.2% | 4.2% | 55.8% | 7.5% |
| 19. | (Stelera-S.pyogens)/S.pyogens | | 19 | 20.4% | 110.6% | 23.2% | 26.8% | | 105.9% | 78.8% | 90.2% | 32.2% |
| 20. | S.Coccus (S.C) | | | 200.39 | 4587.95 | 1831.93 | 662.95 | | 1038.74 | 3419.22 | 567.04 | 8985.92 |
| 20. | Humira | | | 222.11 | 32083.9 | 1961.64 | 718.29 | | 633.97 | 2693.66 | 656.86 | 9421.22 |
| 20. | Stelara | | | 201.66 | 3604.55 | 1971.16 | 658.75 | | 871.31 | 3307.75 | 597.57 | 8574.26 |
| 20. | (Humira-S.Coccus)/S.Coccus | | 20 | 10.8% | 599.3% | 7.1% | 8.3% | | −39.0% | −21.2% | 15.8% | 4.8% |
| 20. | (Stelera-S.Coccus)/S.Coccus | | 20 | 0.6% | −21.4% | 7.6% | −0.6% | | −16.1% | −3.3% | 5.4% | −4.6% |
| 21. | S.Coccus (S.C) | | | 47.44 | 54.91 | 1137.46 | 1690.81 | 1760.88 | 269.18 | 353.15 | 327.98 | 1355.34 |
| 21. | S.Coccus (S.C) + Stelara | | | 40.25 | 52.64 | 1005.07 | 1752.55 | 1944.48 | 241.05 | 408 2 | 327 98 | 1972 66 |
| 21. | (Stelera-S.Coccus)/S.Coccus | | 21 | −15.2% | −4.1% | −11.6% | 3.7% | 10.4% | −10.5% | 15.6% | 0.0% | 45.5% |

To measuring cytokine and chemokine levels, culture supernatants are collected, and the concentrations of IL-6, IP-10, interferon-γ, GM-CSF, PDGF, MCP-1, MIP-la, RANTES, and VEGF are determined using a protein multiplex immunoassay system (Bio-Plex Cytokine Array System, Bio-Rad Laboratories, Hercules, Calif., USA). A ratio (such as the ration estimated by the following formula VII) is calculated in different patients undergoing different biologics in vitro, and the ratio is equal to [(cytokine and chemokine with streptococcus (step d) and biologics) minus (cytokine and chemokine with streptococcus only (step b)]/cytokine and chemokine with streptococcus only (step b). If the patient uses this biological agent and eventually causes paradoxical psoriatic arthritis, it can be classified into the paradoxical psoriatic arthritis group. Conversely, if the patient using another biological agent finally doesn't get paradoxical psoriatic arthritis, it can be classified as a non-paradoxical psoriatic arthritis group

$$Δ RANTES = \frac{step ( d ) RANTES - step ( b ) RANTES}{step ( b ) RANTES} \qquad formula ( VII )$$

TABLE 12
Correlation between Clinical Paradoxical PsA and
non-Paradoxical PsA and Biomarkers among Different Biologies

| | Non-Paradoxical | | Paradoxical PsA | | |
|---|---|---|---|---|---|
| | PsA (n = 44) | | (n = 2) | | |
| | Mean | ± SD | Mean | ± SD | p value |
| IFN-r | −5.3% | 15.8% | −24.1% | 10.3% | 0.069 |
| Hu IP-10 (48) | 19.3% | 99.0% | −13.4% | 24.9% | 0.525 |
| MCP | −17.1% | 66.9% | 15.5% | 8.5% | 0.156 |
| MIP-1α | −3.6% | 36.0% | 12.1% | 21.1% | 0.660 |
| PDGF | −3.7% | 22.8% | −5.4% | 0.6% | 0.891 |
| RANTES | −7.7% | 22.8% | 221.0% | 173.1% | 0.002** |
| VEGF | 1.2% | 20.8% | −16.8% | 16.7% | 0.139 |
| IL-6 | 0.7% | 16.0% | 24.0% | 46.1% | 0.697 |

Mann-Whitney U test.
*p < 0.05,
** p <0.01.

In the above table, the ratio A RANTES is greatly increased and is significantly higher (p=0.002) between after induction with S. pyogenes plus paradoxical PsA-inducing biologics and after induction with S. pyogenes only than after induction with S. pyogenes plus non-paradoxical PsA-inducing biologics and after induction with S. pyogenes only.

In one embodiment, the disqualified drug can be determined by that the expression level of RANTES detected in step (d) is increased by at least 100% than the expression level of RANTES after streptococcus activation in step (b). We can screen in the vitro first and then avoid to use the culprit biologics.

The foregoing description of the preferred embodiments of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners skilled in this art. The embodiments are chosen and described in order to best explain the principles of the invention and its best mode practical application, thereby to enable persons skilled in the art to understand the invention for various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like does not necessarily limit the claim scope to a specific embodiment, and the reference to particularly preferred exemplary embodiments of the invention does not imply a limitation on the invention, and no such limitation is to be inferred. The invention is limited only by the spirit and scope of the appended claims. The abstract of the disclosure is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. It should be appreciated that variations may be made in the embodiments described by persons skilled in the art without departing from the scope of the present invention as defined by the following claims. Moreover, no element and component in the present disclosure is intended to be dedicated to the public regardless of whether the element or component is explicitly recited in the following claims.

What is claimed is:

1. A method for screening a therapeutic drug from a plurality of candidate drugs, the therapeutic drug being used for treating psoriasis in an individual, and the method comprising:
(a) isolating and separating fresh peripheral blood mononuclear cells (PBMC);
(b) activating the peripheral blood mononuclear cells with a stimulant to cause the cells to produce interferon-gamma (IFN-γ) and, regulated on activation, normal T cell expressed and secreted (RANTES);
(c) respectively administering the plurality of candidate drugs to the activated peripheral blood mononuclear cells;
(d) detecting expression levels of IFN-γ and RANTES in the peripheral blood mononuclear cells administered with the plurality of candidate drugs;
(e) recognizing and removing a disqualified drug from the plurality of candidate drugs according to a variation degree of the expression level of RANTES; and
(f) screening the therapeutic drug from the plurality of the candidate drugs excluding the disqualified drug according to the expression level of IFN-γ.

2. The screening method as claimed in claim 1, wherein the screened therapeutic drug is selected from top 40% of the candidate drugs ranked in order from having a smallest expression level to a largest expression level of IFN-γ.

3. The screening method as claimed in claim 1, wherein the screened therapeutic drug has a minimum expression level of IFN-γ among all the candidate drugs.

4. The screening method as claimed in claim 1, wherein the disqualified drug is recognized by that the expression level of RANTES detected in step (d) is increased by at least 100% than the expression level of RANTES after streptococcus activation in step (b).

5. The screening method as claimed in claim 1, wherein the stimulant is Gram-positive bacteria, Gram-negative bacteria, or lipopolysaccharide (LPS).

6. The screening method as claimed in claim 5, wherein the gram-positive bacteria is Streptococcus pyogenes.

7. The screening method as claimed in claim 5, wherein the gram-negative bacteria is Escherichia coli.

8. The screening method as claimed in claim 1, wherein each of the plurality of drug candidates is selected from the group consisting of oral Acitretin, alefacept, Apremilast, Acitretin Tofacitinib, Baricitinib, Cyclosporine, Dithranol, Etanercept, Infliximab, Methotrexate, Pyridoxine Pimecrolimus, Tacrolimus, ALX-0761, BCD-085, Bimekizumab, Brodalumab, CJM112, CNTO 6785, COVA322, eselizumab Ixekizumab, LY3114062, MSB0010841, NI-1401, Perakizumab, Remtolumab, RG7624, Secukinumab, Vunakizumab, Brazikumab, Guselkumab, Mirikizumab, Risankizumab, Tildrakizumab, Ustec Ustekinumab, Adalimumab, Certolizumab, Etanercept, Golimumab and Infliximab.

9. The screening method as claimed in claim 1, wherein the plurality of candidate drugs comprise adalimumab, golimumab, guselkumab, ixekizumab, secukinumab, brodalumab, etanercept and Guselkumab.

10. A method for screening a therapeutic drug from a plurality of candidate drugs, the therapeutic drug being used for treating psoriasis in an individual, and the method comprising:
(a) isolating and separating fresh peripheral blood mononuclear cells (PBMC);
(b) activating the peripheral blood mononuclear cells with a stimulant to cause the cells to produce interferon-gamma (IFN-γ), interleukin-4 (interleukin-4, IL-4), interleukin-9 (interleukin-9, IL-9) and, regulated on activation, normal T cell expressed and secreted (RANTES);
(c) respectively administering the plurality of candidate drugs to the activated peripheral blood mononuclear cells;
(d) detecting expression levels of IFN-γ, IL-4, IL-9 and RANTES in the peripheral blood mononuclear cells administered with the plurality of candidate drugs;
(e) recognizing and removing a disqualified drug from the plurality of candidate drugs according to a variation degree of the expression level of RANTES; and
(f) screening the therapeutic drug from the plurality of the candidate drugs excluding the disqualified drug according to a ratio of the IFN-γ expression level to the IL-4 expression level or according to a ratio of the IFN-γ expression level to the IL-9 expression level.

11. The screening method as claimed in claim 10, wherein the screened therapeutic drug is selected from top 40% of the candidate drugs ranked in order from having a smallest ratio to a largest ratio of the IFN-γ expression level to the IL-4 expression level, or selected from top 40% of the candidate drugs ranked in order from having a smallest ratio to a largest ratio of the IFN-γ expression level to the IL-9 expression level.

12. The screening method as claimed in claim 10, wherein, among all the candidate drugs, the screened therapeutic drug has a minimum ratio of the IFN-γ expression level to the IL-4 expression level or a minimum ratio of the IFN-γ expression level to the IL-9 expression level.

13. The screening method as claimed in claim 10, wherein the disqualified drug is recognized by that the expression level of RANTES detected in step (d) is increased by at least 100% than the expression level of RANTES after streptococcus activation in step (b).

14. The screening method as claimed in claim 10, wherein the stimulant is Gram-positive bacteria, Gram-negative bacteria, or lipopolysaccharide (LPS).

15. The screening method as claimed in claim 14, wherein the gram-positive bacteria is Streptococcus pyogenes.

16. The screening method as claimed in claim 14, wherein the gram-negative bacteria is Escherichia coli.

17. The screening method as claimed in claim 10, wherein each of the plurality of drug candidates is selected from the group consisting of oral Acitretin, alefacept, Apremilast, Acitretin Tofacitinib, Baricitinib, Cyclosporine, Dithranol, Etanercept, Infliximab, Methotrexate, Pyridoxine Pimecrolimus, Tacrolimus, ALX-0761, BCD-085, Bimekizumab, Brodalumab, CJM112, CNTO 6785, COVA322, eselizumab Ixekizumab, LY3114062, MSB0010841, NI-1401, Perakizumab, Remtolumab, RG7624, Secukinumab, Vunakizumab, Brazikumab, Guselkumab, Mirikizumab, Risankizumab, Tildrakizumab, Ustec Ustekinumab, Adalimumab, Certolizumab, Etanercept, Golimumab and Infliximab.

18. The screening method as claimed in claim 10, wherein the plurality of candidate drugs comprise adalimumab, golimumab, guselkumab, ixekizumab, secukinumab, brodalumab, etanercept and Guselkumab.

19. A method for screening a therapeutic drug from a plurality of candidate drugs, the therapeutic drug being used for treating psoriasis in an individual, and the method comprising:
(a) isolating and separating fresh peripheral blood mononuclear cells (PBMC);
(b) activating the peripheral blood mononuclear cells with a stimulant to cause the cells to produce interferon-gamma (IFN-γ), interleukin-4 (interleukin-4, IL-4), interleukin-9 (interleukin-9, IL-9), interleukin-13 (interleukin-13, IL-13) and, regulated on activation, normal T cell expressed and secreted (RANTES);
(c) respectively administering the plurality of candidate drugs to the activated peripheral blood mononuclear cells;
(d) detecting expression levels of IFN-γ, IL-4, IL-9, IL-13 and RANTES in the peripheral blood mononuclear cells administered with the plurality of candidate drugs;
(e) recognizing and removing a disqualified drug from the plurality of candidate drugs according to a variation degree of the expression level of RANTES, wherein the disqualified drug is recognized by that the expression level of RANTES detected in step (d) is increased by at least 100% than the expression level of RANTES after streptococcus activation in step (b); and
(f) screening the therapeutic drug from the plurality of the candidate drugs excluding the disqualified drug according to a ratio of the IFN-γ expression level to the IL-4 expression level, a ratio of the IFN-γ expression level to the IL-9 expression level, or a ratio of the IFN-γ expression level to the IL-13 expression level.
